(19) United States
(12) Patent Application Publication
(10) Pub. No.: US 2025/0270649 A1
(43) Pub. Date: Aug. 28, 2025

(54) BIOMARKER COMPOSITION FOR EARLY DETECTION AND PROGNOSIS PREDICTION OF LUNG ADENOCARCINOMA THROUGH AVEN GENE AND ITS ASSOCIATED GENES

(71) Applicant: Research & Business Foundation SUNGKYUNKWAN UNIVERSITY, Suwon-si (KR)

(72) Inventors: Hong Sook KIM, Suwon-si (KR); DENGXIA FAN, Suwon-si (KR); Jeong Hee LEE, Suwon-si (KR)

(73) Assignee: Research & Business Foundation SUNGKYUNKWAN UNIVERSITY, Suwon-si (KR)

(21) Appl. No.: 18/954,004

(22) Filed: Nov. 20, 2024

(30) Foreign Application Priority Data
Feb. 26, 2024 (KR) 10-2024-0027256
Jun. 19, 2024 (KR) 10-2024-0079411
Oct. 15, 2024 (KR) 10-2024-0140010
Oct. 15, 2024 (KR) 10-2024-0140011

(51) Int. Cl. C12Q 1/6886; G01N 33/574

(52) U.S. Cl. CPC C12Q 1/6886; G01N 33/57423; C12Q 2600/158

(57) ABSTRACT

The present invention relates to a composition for the diagnosis of lung adenocarcinoma through the expression levels of KRT6A, SLC16A3, CTSL, LDHA, and CDC42EP2, which are genes associated with AVEN gene; a diagnostic kit comprising the same, and a method for prognosis prediction of lung adenocarcinoma. The present invention can determine the prognosis of lung adenocarcinoma through the expression levels of KRT6A, SLC16A3, CTSL, LDHA, and CDC42EP2, which are genes associated with AVEN gene, and it is expected that the present invention can be effectively used for screening of target and therapeutic substances for the development of diagnostic and therapeutic substances for preventing lung adenocarcinoma.

### BACKGROUND OF THE INVENTION

### Field of the Invention

The present invention relates to a method for diagnosing the prognosis of lung adenocarcinoma using the expression levels of AVEN gene and its associated genes (i.e., KRT6A, SLC16A3, CTSL, LDHA, and CDC42EP2), and specifically, the present invention relates to a method for diagnosing or predicting the prognosis that affects the survival outcome of early-stage lung adenocarcinoma by using the overexpression of AVEN.

### Description of the Related Art

Lung cancer is the leading cause of cancer-related deaths worldwide. Lung adenocarcinoma (LUAD) is a subtype of non-small cell lung cancer (NSCLC) that originates from the glandular tissue of the lungs, and is the most common type of lung cancer, accounting for approximately 40% of all NSCLC cases. Recently, there have been significant advances in the diagnosis and treatment of lung adenocarcinoma. For example, targeted therapies have been developed which specifically target genetic changes that promote cancer cell growth in certain patients, and immunotherapy has also emerged as a promising treatment option for lung adenocarcinoma. However, despite advances in diagnostic and therapeutic approaches implemented in clinical trials, these treatments have been shown to benefit only limited patient groups. Therefore, it is necessary to find potential and valuable biomarkers for the diagnosis, prognosis, and treatment of cancer.

Apoptosis, caspase activation inhibitor (AVEN) is a protein that plays an important role in inhibiting apoptosis and promoting cell survival. AVEN specifically binds to B-cell lymphoma-extra-large (Bcl-xL), which is a member of anti-apoptotic Bcl-2 family, thereby maintaining anti-apoptotic activity, and interacts with apoptotic protease activating factor 1 (Apaf-1) to prevent the activation of Apaf-1-mediated caspase. In vivo experiments showed that AVEN knockdown reduced tumor growth and in turn increased apoptosis of hematopoietic neoplasms. Clinical studies have reported that AVEN is overexpressed in patients with acute lymphoblastic leukemia/lymphoma and is associated with prognosis (Melzer et al., 2012; Eiβmann et al., 2013). In fact, AVEN expression was found to be significantly higher in relapsed patients (Choi et al., 2006). However, previous studies were limited to blood and bone cancers, and the correlation between AVEN expression and prognosis and immune infiltration in various cancer types still remains unclear.

Under the circumstances, the present inventors have confirmed that AVEN is overexpressed in lung adenocarcinoma cells, and then selected KRT6A, SLC16A3, CTSL, LDHA, and CDC42EP2 as associated genes of the AVEN gene and have confirmed the significance of the prognostic association between these genes and lung adenocarcinoma, and have confirmed that tumor progression and B-cell infiltration were reduced in lung adenocarcinoma patients in whom these genes were overexpressed, and through this association, have provided a method for prognosis prediction of lung adenocarcinoma.

### SUMMARY OF THE INVENTION

The technical problem to be solved by the present invention is to provide a composition and a kit for diagnosing lung adenocarcinoma.

Additionally, the technical problem to be solved by the present invention is to provide a method for providing information for the diagnosis of lung adenocarcinoma.

Additionally, the technical problem to be solved by the present invention is to provide a method for prognosis prediction of lung adenocarcinoma through the expression levels of AVEN gene and associated genes KRT6A, SLC16A3, CTSL, LDHA, and CDC42EP2.

The technical problems to be solved by the present invention are not limited to those mentioned above, and other technical problems not mentioned above will be clearly understood by those skilled in the technical field to which the present invention belongs from the description below.

In order to achieve the above-mentioned technical problems, an embodiment of the present invention provides a biomarker composition for the diagnosis or prognosis prediction of lung adenocarcinoma, which includes a substance that specifically binds to Apoptosis, caspase activation inhibitor (AVEN) mRNA or its protein.

In an embodiment of the present invention, the substance may be at least one selected from the group consisting of antibodies, aptamers, DNA, RNA, proteins, and polypeptides.

In order to achieve the above-mentioned technical problems, another embodiment of the present invention provides a kit for diagnosis or prognosis prediction of lung adenocarcinoma including the composition.

In order to achieve the above-mentioned technical problems, another embodiment of the present invention provides a method for providing information for the diagnosis of lung adenocarcinoma, which includes measuring the expression level of AVEN mRNA or its protein in a sample isolated from a subject for diagnosis.

In an embodiment of the present invention, when the expression level is higher than that of the control group, the method may further include determining that the subject has a higher probability of developing lung adenocarcinoma compared to the control group.

In an embodiment of the present invention, the sample may be selected from the group consisting of tissue, cells, blood, serum, plasma, saliva, and urine.

In an embodiment of the present invention, the prevention or treatment may be by inhibiting the activity in the VEGFA-VEGFR2 pathway activity or increasing immune cells.

In order to achieve the above-mentioned technical problems, the present invention provides a method for prognosis prediction of lung adenocarcinoma, which includes:
a) classifying the gene expression data of a lung adenocarcinoma patient into upper and lower groups of AVEN gene according to the expression level of AVEN;
b) performing an analysis of differentially expressed genes (DEG) for each of a plurality of gene expression data in the two groups;
c) analyzing the correlation between the expression levels of the differentially expressed genes and survival of the lung adenocarcinoma patient;
d) selecting KRT6A, SLC16A3, CTSL, LDHA, and CDC42EP2 genes as AVEN-associated genes; and
e) calculating a prognostic risk score for lung adenocarcinoma based on the expression levels of AVEN gene and the AVEN-associated genes.

The present invention also provides a system for prognosis prediction of lung adenocarcinoma, which includes:
a data preprocessing unit, which classifies the gene expression data of a lung adenocarcinoma patient with lung adenocarcinoma into upper and lower groups of AVEN gene according to the expression level of AVEN;
a gene analysis unit, which performs an analysis of differentially expressed genes (DEG) for each of a plurality of gene expression data in the two selected groups;
a unit of selecting prognostic marker genes for lung adenocarcinoma, which analyzes the correlation between the expression levels of the differentially expressed genes and survival of the lung adenocarcinoma patient;
a unit of determining AVEN-associated genes, which determines KRT6A, SLC16A3, CTSL, LDHA, and CDC42EP2 genes as AVEN-associated genes; and
a unit of calculating the degree of risk, which calculates a prognostic risk score for lung adenocarcinoma based on the expression levels of AVEN gene and the AVEN-associated genes.

In the present invention, the upper group of AVEN gene shows the upper 25% expression level, and the lower group of AVEN gene shows the lower 25% expression level.

In the present invention, the analysis of differentially expressed genes (DEG) may be characterized by selecting genes by examining molecular and cell signaling pathways associated with the AVEN gene through Spearman's Rank Correlation Test.

Additionally, in the present invention, the selection of the prognostic marker genes for lung adenocarcinoma may be characterized by selecting genes with a p value<0.01 through Cox regression analysis as prognostic marker genes for lung adenocarcinoma associated with the survival of patients with lung adenocarcinoma, and may be characterized by calculating the relative importance of prognostic marker genes for lung adenocarcinoma through random survival forest analysis and further selecting the genes with importance values of 0.5 or greater.

Here, the selected prognostic marker genes for lung adenocarcinoma may be selected from the group consisting of KRT6A, SLC16A3, AHNAK2, CTSL, FAM83A, LDHA, CDC42EP2, and SPHK1.

In the present invention, the risk score is calculated using the following formula:

Risk score=h0(t)×exp(KRT6A×0.0002919+SLC16A3×0.0045+CTSL×0.0008009+LDHA×0.007940+CDC42EP2×0.0147).

### Advantageous Effects of the Invention

The present invention relates to a composition for diagnosing lung adenocarcinoma, a diagnostic kit including the same, and a method for providing information for diagnosing lung adenocarcinoma, and it is expected that the composition, kit, and method of the present invention can be usefully utilized for screening of target and therapeutic substances for the development of diagnostic and therapeutic substances for preventing lung adenocarcinoma by using AVEN as a biomarker.

Additionally, the present invention can determine the prognosis of lung adenocarcinoma through the expression level of the AVEN gene and its associated genes (i.e., KRT6A, SLC16A3, CTSL, LDHA, and CDC42EP2), and can provide appropriate treatment plans for lung adenocarcinoma patients by using the same.

The effects of the present invention are not limited to the effects described above, but should be understood to include all effects that can be inferred from the constitutions described in the description or claims of the present invention.

### BRIEF DESCRIPTION OF THE DRAWINGS

FIG. 1A shows the analysis results of the survival of lung adenocarcinoma patients with AVEN^{high} and AVEN^{low} derived from the TCGA database, and FIGS. 1B, 1C, and 1D show the results of expression levels of AVEN mRNA at various stages in lung adenocarcinoma and normal tissues.

FIG. 2A shows the analysis results of pathway enrichment using DEGs as functional signaling pathways related to AVEN expression, and FIG. 2B visualizes the correlation between AVEN expression and genes related to the cell cycle and VEGF-VEGFR2 pathways as a heatmap.

FIG. 3 shows the stromal score, immune score, and estimate score of AVEN^{high} and AVEN^{low} groups.

FIG. 4 shows the immune infiltration environment related to AVEN through immune infiltration estimation using various deconvolution tools including TIMER, MCP-Counter, EPIC, quanTiseq. FIG. 4B presents the immune infiltration estimation results using xCELL, and CIBERSORT.

FIG. 5A is a graph predicting an immune therapy response based on AVEN expression through a bar graph of TIDE scores among 260 patients with different AVEN expression levels, and FIGS. 5B and 5C further demonstrate that NSCLC patients with a high AVEN level tend to have a poor response to immunotherapy.

FIG. 6A shows a random survival forest analysis using DEG, FIG. 6B shows AVEN-derived genes with variable relative importance exceeding 0.5, and FIG. 6C confirms a significant positive correlation between AVEN and five genes derived from the same (i.e., KRT6A, SLC16A3, CTSL, LDHA, and CDC42EP2) between the upper and lower groups of AVEN.

FIG. 7 relates to an AVEN-derived prognosis model of lung adenocarcinoma, in which FIG. 7A shows the survival analysis between lung adenocarcinoma patients with a high-risk score and those with a low-risk score using TCGA lung adenocarcinoma data. FIG. 7B presents a time-dependent ROC curve based on the optimized AVEN-derived prognosis model. FIGS. 7C and 7D show the correlation between the risk score calculated according to the present invention and the survival rate of lung adenocarcinoma patients.

### DETAILED DESCRIPTION OF THE EMBODIMENTS

Hereinafter, the present invention will be described

The present invention relates to a composition for the diagnosis of lung adenocarcinoma.

The composition of the present invention includes a substance that specifically binds to AVEN mRNA or its protein.

Apoptosis, caspase activation inhibitor (AVEN) is a protein that plays an important role in inhibiting apoptosis and promoting cell survival. AVEN specifically binds to B-cell lymphoma-extra-large (Bcl-xL), which is a member of anti-apoptotic Bcl-2 family, thereby maintaining anti-apoptotic activity, and interacts with apoptotic protease activating factor 1 (Apaf-1) to prevent the activation of Apaf-1-mediated caspase. AVEN gene has no known function in regulating lung adenocarcinoma growth in lung adenocarcinoma cells. Nothing has been known regarding the use of AVEN gene with respect to the regulation of growth of lung adenocarcinoma in lung adenocarcinoma cells.

AVEN is present in a sample derived from a subject for diagnosis, and for its mRNA or protein sequence, those published in the NCBI genebank, etc. may be used. For example, the sequences of the individual species that is to be a subject for diagnosis.

The subject for diagnosis is an animal that currently has lung adenocarcinoma, has had lung adenocarcinoma, or is an animal for which it is desired to obtain the information for predicting or diagnosing the occurrence of lung adenocarcinoma. The animal may be a mammal, including a human.

The sample is isolated from a subject for diagnosis, and may be, for example, tissue, cells, blood, serum, plasma, saliva, or urine, and specifically, may be cancer tissue isolated from a subject for diagnosis, but is not limited thereto.

The substance is not limited as long as it can detect AVEN mRNA or its protein, but may be at least one selected from the group consisting of, for example, antibodies, aptamers, DNA, RNA, proteins, and polypeptides.

As used herein, “antibody” means a protein molecule specific for an antigenic site. For the purposes of this application, antibody means an antibody that specifically binds to AVEN, which is a marker protein, and may include all of monoclonal antibodies, polyclonal antibodies, and recombinant antibodies.

The monoclonal antibody may be prepared using a hybridoma method or phage antibody library technology widely known in the art, but the method may not be limited thereto.

The polyclonal antibodies may be prepared by methods well known in the art, including injecting the protein antigen into an animal and collecting blood from the animal to obtain serum including antibodies. These polyclonal antibodies may be prepared from any animal species host, including goats, rabbits, sheep, monkeys, horses, pigs, cow, dogs, etc., but may not be limited thereto.

Additionally, the antibodies of the present invention may also include special antibodies such as chimeric antibodies and humanized antibodies.

The “peptide” has the advantage of high binding affinity to a target substance and does not undergo denaturation even when subjected to heat/chemical treatment. Additionally, due to a small molecular size, it can be used as a fusion protein by attaching it to another protein. Specifically, since it can be used by attaching it to a polymer protein chain, it can be used as a diagnostic kit and a drug delivery material.

The “aptamer” refers to a type of polynucleotide consisting of a special type of single-stranded nucleic acid (DNA, RNA, or a modified nucleic acid) that has a stable tertiary structure in itself and has the characteristic of being able to bind to a target molecule with high affinity and specificity. As described above, aptamers can specifically bind to antigenic substances in the same way as antibodies, but are more stable than proteins, have a simple structure, and consist of polynucleotides that are easy to synthesize, and thus can be used as a substitute for antibodies.

The substance that specifically binds to the mRNA may be sense and antisense primers, or probes, but is not limited thereto.

In the present invention, “primer” refers to a short genetic sequence that serves as the starting point for DNA synthesis, and is an oligonucleotide synthesized for the purpose of diagnosis, DNA sequencing, etc. The primers may be synthesized in a length of 15 to 30 base pairs for use, but the length may vary depending on the intended use, and may be modified by methylation, capping, etc. using known methods.

In the present invention, the term “probe” refers to a nucleic acid that can specifically bind to mRNA of a few bases to several hundred bases in length, prepared through an enzymatic chemical separation, purification or synthetic process. The presence or absence of mRNA can be confirmed by labeling with radioactive isotopes or enzymes, and can be designed and modified using known methods.

As for a probe or primer that specifically binds to a nucleotide sequence of the gene encoding the AVEN, a sequence complementary to the nucleotide sequence, or a fragment of the nucleotide sequence, the nucleotide sequence of the gene encoding the AVEN is known; therefore, those skilled in the art can design the primer or probe based on the sequence according to a conventional method in the art.

The composition of the present invention is a substance for use in predicting or diagnosing lung adenocarcinoma in patients suspected of lung adenocarcinoma, and the present invention can be used to predict or diagnose lung adenocarcinoma in a suspected patient by processing a sample isolated from a subject for diagnosis and measuring the expression level of AVEN mRNA or protein in the sample.

For example, when the expression level of AVEN mRNA or its protein in a sample of a patient suspected of having lung adenocarcinoma is compared with the expression level of a normal control, and the expression level of the patient suspected of having lung adenocarcinoma is higher than that of the normal control, it may be determined that the probability of occurrence of lung adenocarcinoma in the patient suspected of having lung adenocarcinoma is higher compared to the normal control group.

The present invention relates to a kit for diagnosing lung adenocarcinoma including the composition.

The kit may include not only a substance that specifically binds to AVEN mRNA or its protein, but also one or more other compositions with different components, solutions, or devices suitable for an analytical method for measuring the level of AVEN mRNA or its protein expression used by the kit.

When the kit is a kit for measuring the expression level of AVEN mRNA or its protein, it may be a kit including essential elements required for performing RT-PCR. In addition to each primer pair specific for the mRNA of the marker gene, the RT-PCR kit may include a test tube or other appropriate containers, reaction buffer, deoxyribonucleotides (dNTPs), enzymes such as Taq polymerase and reverse transcriptase, DNase and RNase inhibitors, DEPC water, sterile water, etc. Additionally, the RT-PCR kit may include a primer pair specific for the gene used as a quantitative control.

For immunological detection of substances that specifically bind to a nucleotide sequence of the gene encoding AVEN, a sequence complementary to the nucleotide sequence, a fragment of the nucleotide sequence, or a protein encoded by the nucleotide sequences, the kit may include a substrate, a suitable buffer, a secondary antibody labeled with a chromogenic enzyme or fluorescent substance, and a chromogenic substrate. As the substrate, a nitrocellulose membrane, a 96-well plate synthesized with polyvinyl resin, a 96-well plate synthesized with polystyrene resin, glass slide glasses, etc. may be used; as chromogenic enzyme, peroxidase and alkaline phosphatase may be used; and as the chromogenic substrate, 2,2′-azino-bis(3-ethylbenzothiazoline-6-sulfonic acid) (ABTS) or o-phenylenediamine (OPD), tetramethylbenzidine (TMB), etc. may be used.

The kit may be a microarray capable of measuring the expression level of AVEN mRNA. The microarray can easily be prepared by those skilled in the art using the index factors according to a method known in the art. According to a specific embodiment, it may be a microarray in which a cDNA of a sequence corresponding to an mRNA of a gene encoding the AVEN protein or a fragment thereof is attached to a substrate as a probe.

Additionally, the kit of this application may include an antibody that specifically binds to a marker component, a secondary antibody conjugate to which a label that develops color by a reaction with a substrate is conjugated, a chromogenic substrate solution to be chromogenically reacted with the label, a washing solution, and an enzyme reaction stop solution, etc., and may be prepared into a number of separate packaging or compartments including the reagent components to be used, but may not be limited thereto.

The kit of this application may include not only a formulation capable of measuring the expression level of AVEN mRNA or its protein in a patient sample, but also one or more compositions, solutions, or devices suitable for analyzing the expression level. For example, for immunological detection of an antibody, the kit may include a substrate, a suitable buffer, a secondary antibody labeled with a detection label, a chromogenic substrate, etc., but may not be limited thereto.

In a specific embodiment, the kit may be an ELISA kit characterized by including essential elements necessary for performing ELISA to implement various ELISA methods, such as sandwich ELISA. Such an ELISA kit includes specific antibodies against the above-identified proteins. These antibodies are antibodies with high specificity and affinity for AVEN and little cross-reactivity to other proteins, and may be monoclonal, polyclonal, or recombinant antibodies. Additionally, the ELISA kit may include antibodies specific for a control protein. Other ELISA kits may include reagents capable of detecting bound antibodies (e.g., labeled secondary antibodies, chromophores, enzymes and their substrates, or other substances capable of binding to antibodies), but are not limited thereto.

In addition, the kit may be a kit for implementing Western blot, immunoprecipitation analysis, complement fixation analysis, flow cytometry, protein chip, etc. and may further include additional components suitable for each analysis method. Through these analysis methods, anticancer drug resistance can be diagnosed by comparing the amount of an antigen-antibody complex formed.

The present invention relates to a method for providing information for diagnosing lung adenocarcinoma.

The method of the present invention includes measuring the expression level of AVEN mRNA or its protein in a sample isolated from a subject for diagnosis.

The measurement may be performed by treating the sample with a substance that specifically binds to AVEN mRNA or its protein.

The substance that specifically binds to the AVEN mRNA or its protein may be within the range exemplified above.

The sample and the subject for diagnosis are as described above.

As a method for measuring the expression level of the AVEN mRNA or its protein, a method may be selected in which measure the concentration of mRNA, which is a transcript of the gene encoding AVEN, in a sample or the concentration of the AVEN protein in a sample, but is not limited thereto, and may be performed by selecting a method commonly used in the technical field of the present invention.

The methods for measuring the concentration of the above mRNA in a sample may include reverse transcriptase polymerase reaction (RT-PCR), competitive RT-PCR, real-time RT-PCR, RNase protection assay (RPA), Northern blotting, DNA chips, etc., but are not limited thereto.

As a method for measuring the concentration of the protein in a sample, the amount of the protein can be confirmed using an antibody that specifically binds to the protein. The analysis methods for this purpose may include immunoblot assay, sandwich assay, enzyme linked immunosorbent assay (ELISA), radioimmunoassay (RIA), radioimmunodiffusion, Ouchterlony immunodiffusion method, rocket immunoelectrophoresis, immunohistochemistry, immunoprecipitation assay, complement fixation assay, fluorescence-activated cell sorting (FACS), Western blotting, flow cytometry, an enzyme-substrate color development method, antigen-antibody agglutination, protein chip, etc., but are not limited thereto.

The method of the present invention may further include comparing the above-identified expression level with that of a control group.

After comparing the expression level of AVEN mRNA or its protein in a sample from a patient suspected of having lung adenocarcinoma with that of a normal control patient, if the expression level of AVEN in the patient suspected of having lung adenocarcinoma is higher than that in a normal control patient, the patient suspected of having lung adenocarcinoma may be determined to have a high probability of developing lung adenocarcinoma. Additionally, when the expression level of the AVEN mRNA or its protein is measured to have no statistically significant difference (e.g., p<0.05) or to be lower than that of a normal control patient, it is possible to provide information that can help determine that the patient suspected of having lung adenocarcinoma has a low risk of developing lung adenocarcinoma.

Additionally, after comparing the expression levels of AVEN mRNAs or their proteins in samples from two patients with suspected of having lung adenocarcinoma were compared, in the case of the patient with a higher expression level, the patients with a lower expression level may be determined to have a higher risk of developing lung adenocarcinoma than that with a higher expression level.

Additionally, after comparing the expression level of AVEN mRNA or its protein in a sample from a patient suspected of having lung adenocarcinoma with that of a lung adenocarcinoma patient, if the expression level of patients suspected of lung adenocarcinoma has no statistically significant difference from the lung adenocarcinoma patient (P<0.05), it may be determined that there is a high possibility of developing lung adenocarcinoma.

The present invention relates to a method for screening for a therapeutic agent for lung adenocarcinoma.

The method of the present invention includes treating a candidate substance to a biological sample expressing AVEN mRNA or its protein; measuring the expression level of AVEN mRNA or its protein in a sample treated with the candidate substance; and selecting a candidate substance whose expression level is reduced compared to that of a control group not treated with the candidate substance.

The method for treating a candidate substance in a biological sample is not particularly limited, and may be treating a candidate substance in tissue, cells, blood, serum, plasma, saliva, or urine isolated from an individual.

The expression level of AVEN mRNA or its protein may be measured by the method described above.

The present invention has confirmed that the expression level of AVEN gene is increased in lung adenocarcinoma cells, and lung adenocarcinoma is cured when the expression level of AVEN gene is decreased, and when the candidate substance is treated with a biological sample expressing AVEN mRNA or its protein and as a result, the expression level of AVEN mRNA or its protein in the sample decreases, the candidate substance may be selected as a therapeutic agent for lung adenocarcinoma.

This selection may be performed by measuring the expression level of AVEN mRNA or its protein in a sample before treating the candidate substance to a biological sample, and comparing the expression level after treatment; or the candidate substance may be treated to several samples and the expression levels may then be compared to that of the control group.

The candidate substance refers to an unknown substance used in screening to examine whether it affects the expression level of AVEN mRNA or its protein. The candidate substances include chemicals, nucleotides, antisense RNA, small interference RNA (siRNA), and extracts of natural products, but are not limited thereto.

According to the present invention, there is provided a method for prognosis prediction of lung adenocarcinoma, which includes a) classifying the gene expression data of a lung adenocarcinoma patient with lung adenocarcinoma into upper and lower groups of AVEN genes according to the expression level of AVEN; b) performing an analysis of differentially expressed genes (DEG) for each of a plurality of gene expression data in the two groups; c) analyzing the correlation between the expression levels of the differentially expressed genes and survival of the lung adenocarcinoma patient; d) selecting KRT6A, SLC16A3, CTSL, LDHA, and CDC42EP2 genes as AVEN-associated genes; and e) calculating a prognostic risk score for lung adenocarcinoma based on the expression level of AVEN gene and the AVEN-associated genes.

The present invention also provides a system for prognosis prediction of lung adenocarcinoma, which includes: a data preprocessing unit, which classifies the gene expression data of a lung adenocarcinoma patient into upper and lower groups of AVEN gene according to the expression level of AVEN; a gene analysis unit, which performs an analysis of differentially expressed genes (DEG) for each of a plurality of gene expression data in the two selected groups; a unit of selecting prognostic marker genes for lung adenocarcinoma, which analyzes the correlation between the expression levels of the differentially expressed genes and survival of the lung adenocarcinoma patient; a unit of determining AVEN-associated genes, which determines KRT6A, SLC16A3, CTSL, LDHA, and CDC42EP2 genes as AVEN-associated genes; and a unit of calculating the degree of risk, which calculates a prognostic risk score for lung adenocarcinoma based on the expression levels of AVEN gene and the AVEN-associated genes.

In the present invention, mRNA analysis of AVEN-associated genes in the upper and lower groups was performed, and thereby confirmed that there is a significant positive correlation between AVEN and five genes derived from the same (i.e., KRT6A, SLC16A3, CTSL, LDHA, and CDC42EP2). In the present invention, in order to optimize the prognosis prediction model of lung adenocarcinoma, stepwise forward multivariate Cox analysis was used to further screen prognostic genes including KRT6A, SLC16A3, CTSL, LDHA, and CDC42EP2. First, the initial model was started without any prediction variables, and then each prediction variable was added to the model and evaluated one by one, and the prediction variable that improved the model the most was selected according to the Akaike Information Criterion (AIC). Models with lower AIC values are preferred because they represent a better balance between model fit and complexity. This method led to the identification of the best combination including KRT6A, SLC16A3, AHNAK2, CTSL, FAM83A, LDHA, and CDC42EP2 for the prognosis prediction of lung adenocarcinoma.

The risk score of each patient was calculated by the risk score equation according to the model, and was classified into two groups: high score and low score according to the average risk score. Then, a survival analysis was performed to evaluate the survival of patients with high and low risk scores, and as expected, patients with a high risk score were shown to have a shorter life expectancy, whereas those with a low risk score were shown to have an improved survival rate.

Hereinafter, the present invention will be described in detail by way of examples to provide specific explanations.

### <Example 1> Experimental Materials and Methods

### 1. Pan-Cancer Analysis and TCGA Data Processing

The expression of AVEN mRNA in pan-Cancer was analyzed using the GSCA web tool (http://bioinfo.life.hust.edu.cn/GSCA). For the survival analysis in colon adenoma (COAD), kidney renal clear cell carcinoma (KIRC), kidney renal papillary cell carcinoma (KIRP), lung adenocarcinoma (LUAD), lung squamous cell carcinoma (LUSC), and thyroid carcinoma (THCA), the GEPIA (Li et al., 2021) (http://gepia.cancer-pku.cn/) web tool was applied. In order to further examine the role of AVEN in lung adenocarcinoma, the RNA-sequencing data and clinical data of lung adenocarcinoma patients derived from the TCGA database were obtained from the UCSC Xena website (http://xena.ucsc.edu/). The Log2 (FPKM+1) values obtained from RNA-sequencing data were converted to transcripts per million (TPM) values. Thereafter, patients with the highest 25% of AVEN expression were classified into the AVEN^{high} group, and patients with the lowest 25% of AVEN expression were classified into the AVEN^{low} group. The overall survival analysis was performed in the AVEN^{high} and AVEN^{low} groups.

### 2. Analysis of Characteristics of Lung Adenocarcinoma Patients

In order to compare the characteristics between AVEN^{high} and AVEN^{low} patients, clinical data including age, TNM classification, sex, radiotherapy status, race, AVEN expression, and smoking status were downloaded from the UCSC Xena website. Patient characteristics between the AVEN^{high} and AVEN^{low} groups were visualized using the R package moonBook.

### 3. Analysis of Driver Gene Mutations

In order to examine genetic mutations, whole exome data of lung adenocarcinoma patients were downloaded from cBioportal (https://www.cbioportal.org/). Driver genes such as TP53, EGFR, KRAS, ERBB2, BRAF, ALK, RET, FGFR3, NTRK3, and ROS1 were selected, and their mutation patterns were examined in AVEN^{high} and AVEN^{low} lung adenocarcinoma patients. Oncoprint graphs were generated using the R package ComplexHeatmap.

### 4. Identification of DEGs and Functional Enrichment Analysis

In order to obtain differentially expressed genes (DEGs) between AVEN^{high} and AVEN^{low} groups, the Spearman rank correlation test was used, and the genes with absolute R-values>0.4 were used for pathway analysis by Consensus PathDB (http://cpdb.molgen.mpg.de/MCPDB). Significantly altered pathways were selected with a criterion of p<0.05 and visualized using SRplot (https://www.bioinformatics.com.cn/srplot). Genes in the cell cycle and VEGFA-VEGFR2 pathways were visualized in more detail using the R package ComplexHeatmap. The gene set enrichment analysis (GSEA) analysis was performed to compare pathway enrichment between AVEN^{high} and AVEN^{low} groups, and these gene sets were used as reference: (see: “SHEDDEN_LUNG_CANCER_POOR_SURVIVAL_A6”, “HallMARK_MTORC1_SIGNALINF”, “HALLMARK_EPITHELIAL_MESENCHYMAL_TRANSITION” and “VEGF_A_UP.V1_DN”).

### 5. Analysis of Immune Infiltration

Estimate scores representing immune infiltration, stromal score, and tumor purity were obtained from the Estimate website (https://bioinformatics.mdanderson.org/estimate/index.html) and compared between AVEN^{high} and AVEN^{low} patients. Additionally, several decellularization tools were used to examine different types of immune cells (e.g., Timer, CIBERSORT, EPIC, xCEll, Quanti-seq, and MCP-counter) in tumor tissues. Immune infiltration data were obtained from Timer (https://cistrome.shinyapps.io/timer/) and visualized using boxplots via the R package ggplot2.

### 6. Prediction of Immunotherapy Response

In order to evaluate the prediction value of AVEN in an immunotherapy response, the Tumor Immune Dysfunction And Exclusion score (TIDE) was calculated (http://tide.dfci.harvard.edu/). As a result, the expression levels of immune checkpoint genes and functional genes related to cytotoxic T cells in AVEN^{high} and AVEN^{low} lung adenocarcinoma were to analyzed. Further, the examination include additional immunotherapy response markers representing B cells was expanded, and these markers were tested in patients with AVEN^{high} and AVEN^{low} lung adenocarcinoma. Additionally, the prediction efficiency of AVEN for an immunotherapy response was verified using RNAseq and patient data obtained from two cohorts (GSES207422, GSE135222) that received the immunotherapy. The ICI response between AVEN^{high} and AVEN^{low} NSCLC patients was evaluated using stacked bar charts and survival plots using the R package ggplot2.

### 7. Construction of AVEN-Based Prognostic Genetic Model

In order to determine the correlation between each gene of DEGs and the overall survival of lung adenocarcinoma patients, Univariate Cox regression models were used. AVEN-derived genes with p-value<0.01 were considered AVEN-derived prognostic factors. The relative importance of each gene was calculated using Random Survival Forest analysis. Genes with relative importance>0.5 were used in a Multivariate Cox regression model. Step forward Cox regression was used to optimize the model, and the AVEN-derived genomic model was formulated as follows.

$$Risk score = h 0 ( t ) \times exp ( KRT 6 A \times 0.0002919 + SLC 16 A 3 \times 0.0045 + CTSL \times 0.0008009 + LDHA \times 0.00794 + CDC 42 EP 2 \times 0.0147 )$$

h0(t) represents the individual baseline risk when all prediction variables are set to 0, and patients were classified into high-risk and low-risk groups based on the calculated risk In order to test the sensitivity and specificity of the score. AVEN-derived prognostic genetic model, ROC curves were generated and the test was performed using the survivalROC package.

Additionally, the expression of five prediction genes (i.e., KRT6A, SLC16A3, CTSL, LDHA, and CDC42EP2) was analyzed in AVEN^{high} and AVEN^{low} patients.

### 8. Web-Based Analysis of Bioinformatics

In order to assess the prognostic significance of AVEN in various lung adenocarcinoma data cohorts, PrognoScan was used (Mizuno et al., 2009). Additionally, AVEN protein abundance was examined using cProSite (Wang et al., 2023).

### 9. Statistical Analysis

Data were analyzed using the R software package (v4.2.1). In the present invention, comparisons between groups were performed using Student's t-test, and interactions between variables were examined using the Spearman's correlation analysis.

### <Example 2> Experimental Results

### 1. Confirmation of Correlation Between High Levels of AVEN and Decreased Survival Rate of Lung Adenocarcinoma (Use of TNM System)

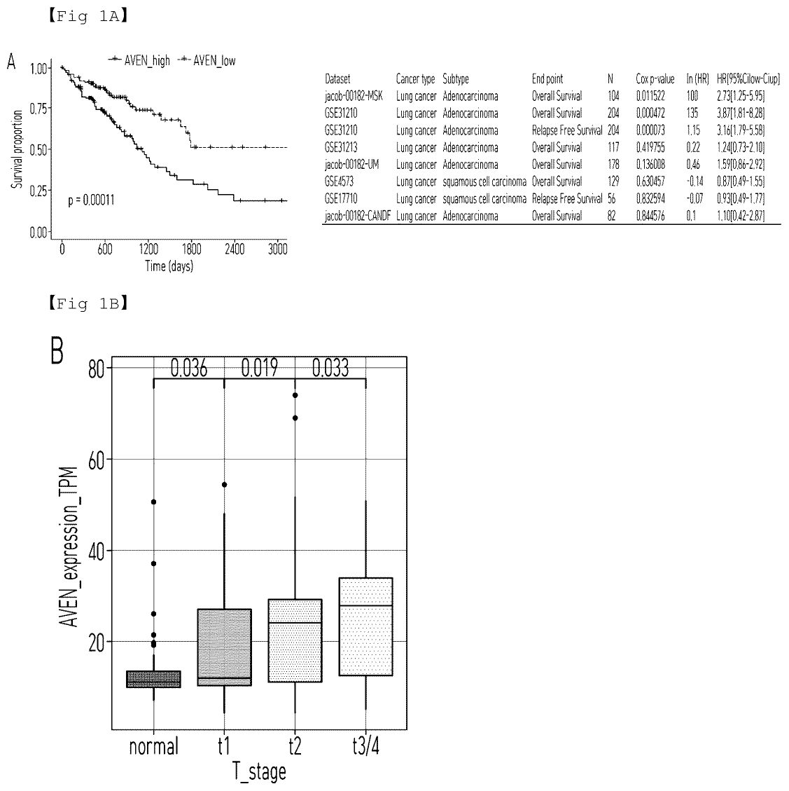

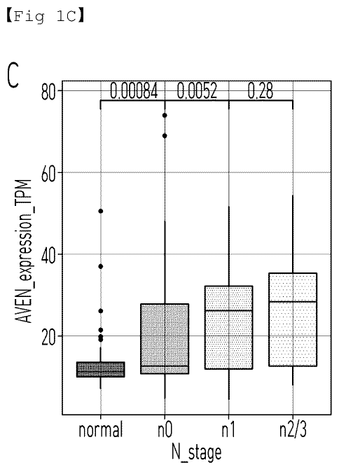

AVEN was overexpressed in six types of cancer, including colon adenoma (COAD), kidney renal clear cell carcinoma (KIRC), kidney renal papillary cell carcinoma (KIRP), lung adenocarcinoma (LUAD), lung squamous cell carcinoma (LUSC), and thyroid adenocarcinoma (THCA), compared with normal tissue (FIG. 1A). In particular, AVEN showed a significant prognostic effect in lung adenocarcinoma.

Upon review of the results of mRNA sequencing data and clinical information obtained from lung adenocarcinoma patients from the TCGA dataset in FIG. 1B, lung adenocarcinoma patients were classified into two groups: AVEN^{high} (upper 25%) and AVEN^{low} (lower 25%), and overall survival was examined using the Kaplan-Meier analysis based on AVEN expression levels. These results showed that high AVEN expression in lung adenocarcinoma was significantly associated with a decreased survival rate.

The TNM classification is a system that defines the tumor size (T), regional lymph node volume (N), and spread (M) of cancer in the patient's body (Rosen and Sapra, 2022). As shown in Table 1 below, AVEN expression did not show significant differences in age, race, and smoking, but only in sex, and particularly, the values of the T stage and N stage showed significant differences between the two groups.

AVEN expression was shown to increase in a tumor-dependent manner when the tumor size and T stage differed from those of normal tissue (FIG. 1C). AVEN expression increased at stage n1 compared to n0 (FIG. 1D). Additionally, AVEN expression was analyzed at various M stages, but there was no significant difference in the number of patients at various M stages between AVEN^{high} and AVEN^{low} patients, but the AVEN expression was similar between M0 and M1 stages (FIG. 1E).

TABLE 1

| | | |
|---|---|---|
| AVEN^{high} | AVEN^{low} | |
| (N = 130) | (N = 130) | p |

| | | | |
|---|---|---|---|
| Age (years) | 65.1 ± 9.7 | 64.5 ± 10.5 | 0.624 |

| | | | | | |
|---|---|---|---|---|---|
| T Stage | | | | | 0.03 |
| t1 | 31 | (23.1%) | 55 | (41.9.0%) | |
| t2 | 75 | (57.2%) | 66 | (50.3%) | |
| t3 | 15 | (11.5%) | 8 | (6.1%) | |
| t4 | 9 | (6.9%) | 1 | (0.8%) | |
| uncharacterized | 1 | (0.8%) | 1 | (0.8%) | |
| N Stage | | | | | 0.001 |
| n0 | 67 | (51.1%) | 93 | (71.5%) | |
| n1 | 33 | (25.2%) | 17 | (13.1%) | |
| n2 | 27 | (20.6%) | 11 | (8.5%) | |
| n3 | 0 | (0.0%) | 1 | (0.8%) | |
| uncharacterized | 4 | (3.1%) | 8 | (6.2%) | |
| M Stage | | | | | 0.061 |
| m0 | 92 | (70.8%) | 84 | (65.6%) | |
| m1 | 11 | (8.4%) | 5 | (3.8%) | |
| uncharacterized | 27 | (20.8%) | 39 | (30.5%) | |
| Sex | | | | | 0.047 |
| Female | 62 | (47.3%) | 79 | (60.3%) | |
| Male | 69 | (52.7%) | 52 | (39.7%) | |
| Radiation | | | | | 0.731 |
| therapy | | | | | |
| no | 98 | (83.8%) | 106 | (86.2%) | |
| Yes | 19 | (16.2%) | 17 | (13.8%) | |
| Race | | | | | 0.72 |
| Asian | 3 | (2.7%) | 3 | (2.6%) | |
| Black or | 9 | (8.0%) | 13 | (11.2%) | |
| African | | | | | |
| American | | | | | |
| White | 100 | (89.3%) | 100 | (86.2%) | |

| | | | |
|---|---|---|---|
| AVEN expression | 31.9 ± 8.5 | 10.5 ± 2.0 | 0 |

| | | | | | |
|---|---|---|---|---|---|
| (TPM) | | | | | |
| Smoke | | | | | 0.788 |
| no | 90 | (68.7%) | 93 | (71.0%) | |
| yes | 41 | (31.3%) | 38 | (29.0%) | |

2. Identification of Functional Signaling Pathways Associated with AVEN

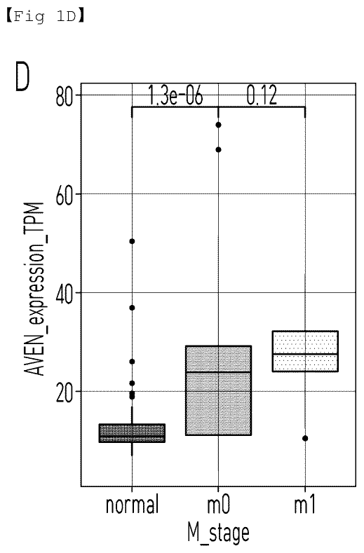

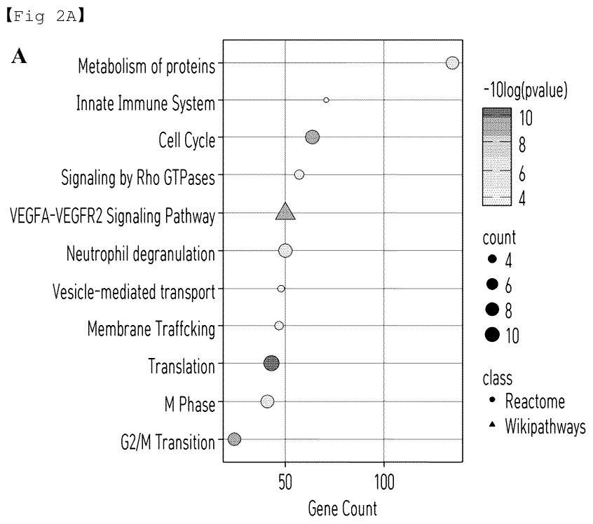

As a result of examination of molecular and cell signaling pathways associated with AVEN using the Spearman's rank correlation analysis, a total of 838 genes were obtained, whereas as a result of performing path enrichment analysis using the ConsensusPathDB website tool, it was found that these DEGs are mainly involved in biological process pathways, oncogenesis pathways (cell cycle and VEGFA-VEGFR2 signaling pathway), and immune regulatory processes (see FIG. 2A).

It was confirmed that AVEN enhances tumor aggressiveness by promoting cell cycle and angiogenesis, and is associated with a cell cycle or VEGFA-VEGFR2 signaling pathway. As shown in FIG. 2B, genes belonging to the MTORC, epithelial-mesenchymal-transition (EMT), and VEGF pathways showed a significant enrichment in AVEN^{high} patients.

3. Confirmation of Immune Infiltration Analysis Associated with AVEN in Lung Adenocarcinoma

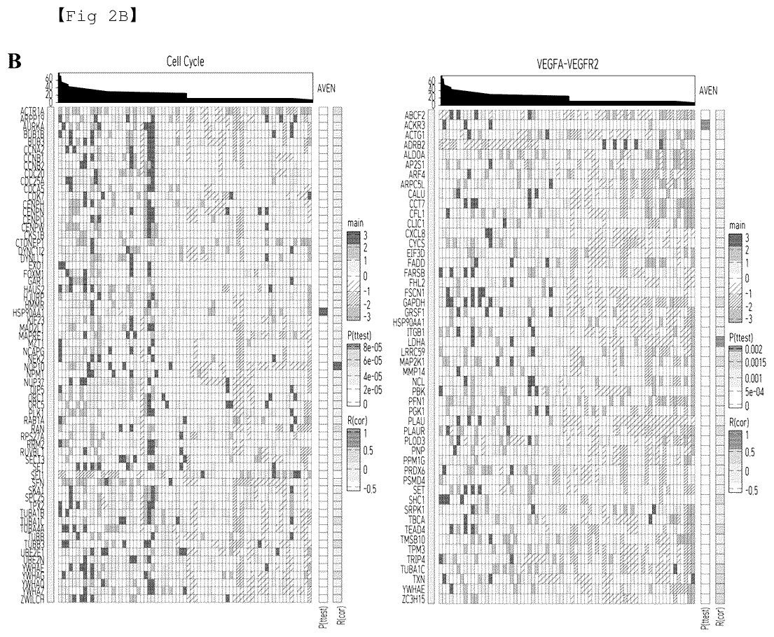

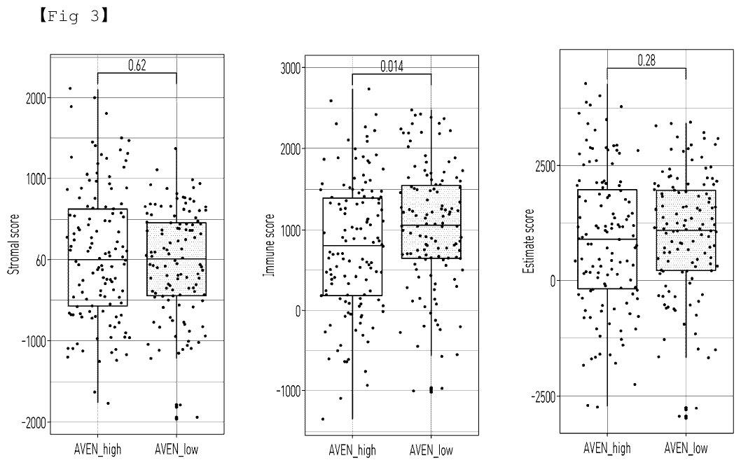

As shown in FIG. 2B, signaling pathways associated with AVEN include pathways such as the innate immune system, neutrophil degranulation, and EMT, thereby suggesting unique TME features associated with AVEN expression. Therefore, first, as a result of the analysis of immune cells and phenotype cells using the ESTIMATE algorithm in the AVEN^{high} and AVEN^{low} groups, AVEN^{high} patients showed lower immune scores than AVEN^{low} patients, this suggests that AVEN has the functional potential to inhibit the enrichment of immune cells within the TME. Additionally, the AVEN^{high} and AVEN^{low} group was shown to have a similar tumor purity (FIG. 3).

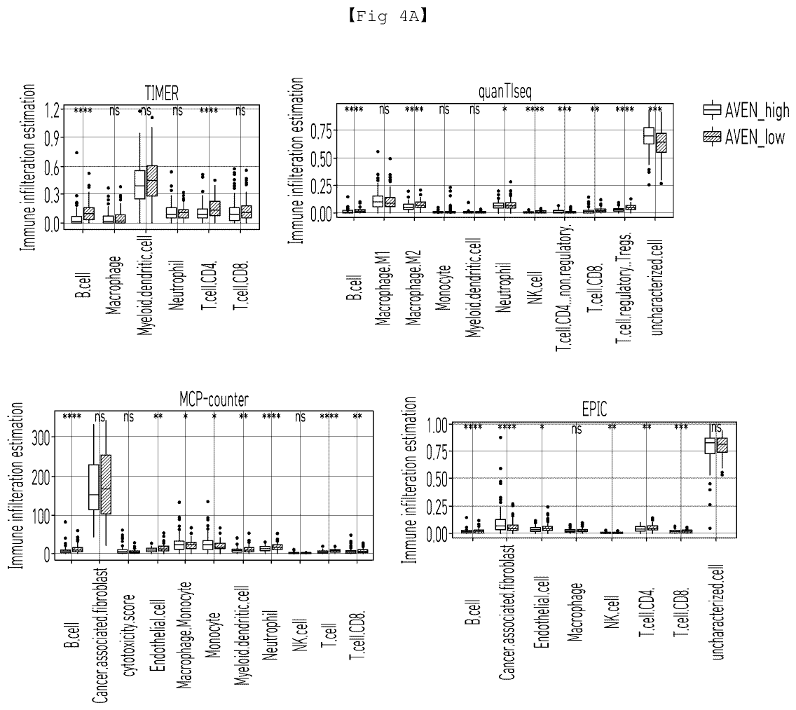

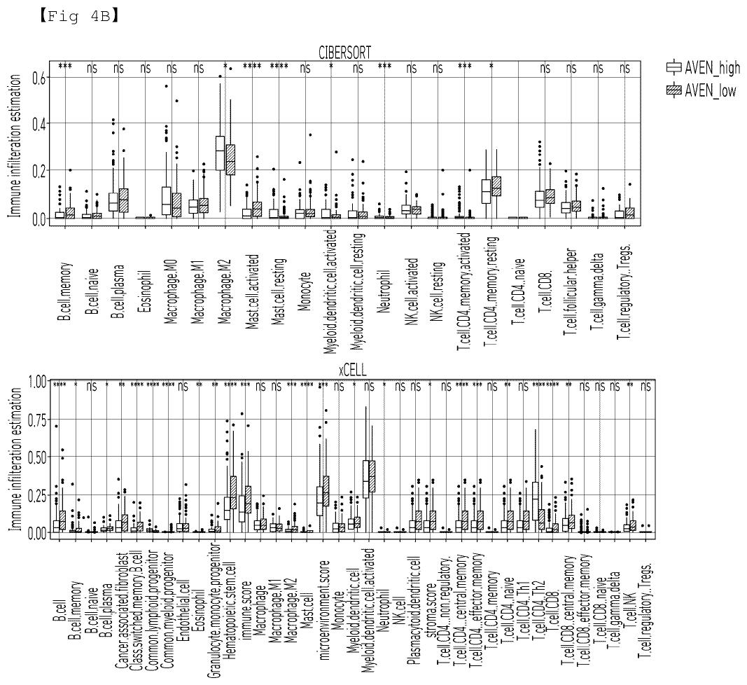

As shown in FIG. 4, as a result of the exploration of different types of immune cells using several deconvolution tools such as Timer, CIBERSORT, EPIC, xCEll, Quanti-seq, and MCP-counter, it was found that B cells were significantly more enriched in the AVEN^{low} group. Timer and EPIC revealed that CD4 T cells were significantly higher in the AVEN^{low} group, whereas Quanti-Seq revealed that the CD4 T cells were higher in the AVEN^{high} group. Additionally, CIBERSORT analysis revealed that activated memory CD4 T cells were more enriched in the AVEN^{high} group, whereas resting CD4 T cells were higher in the AVEN^{low} group (FIG. 4). EPIC, MCP-counter, and xCEll remove tumor-associated fibroblasts (CAFs), and specifically, EPIC and MCP-counter revealed that CAFs were significantly more enriched in the AVEN^{high} group. However, contrary to these results, the xCEll tool revealed that CAFs were shown to be lower in the AVEN^{high} group (FIG. 4).

### 4. Prediction of Immunotherapy Response Through AVEN's Hypersensitivity

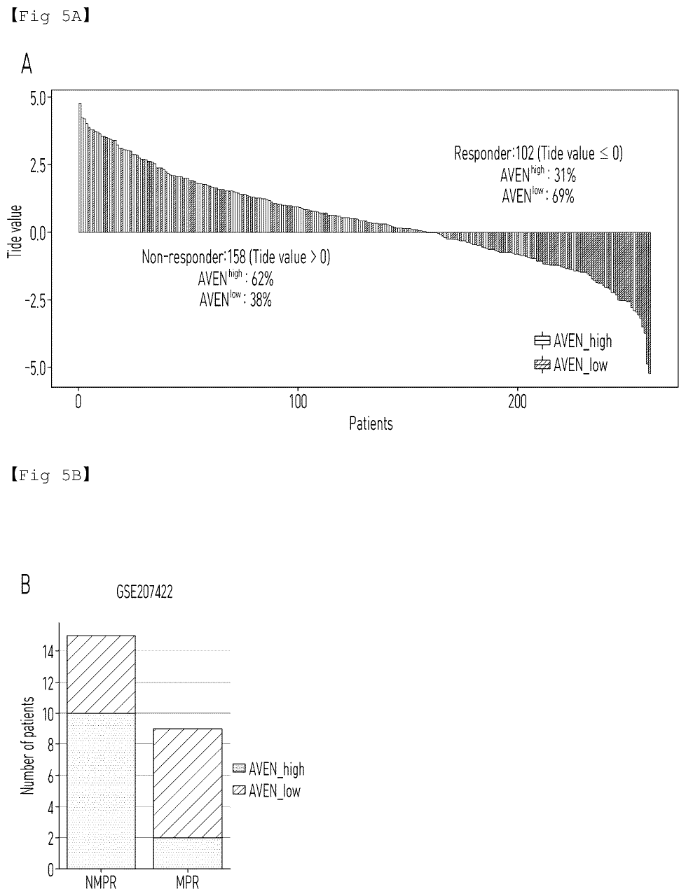

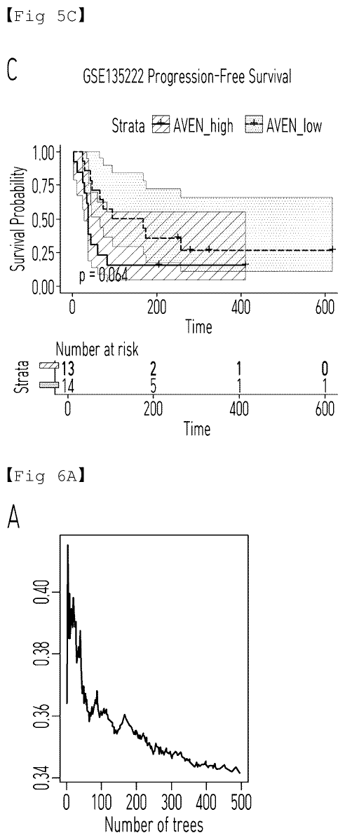

In order to explore the immunotherapy response associated with AVEN, RNA-seq data from 260 patients in the AVEN^{high} and AVEN^{low} groups were processed in the TIDE tool. As a result, among the 158 non-responsive patients, 62% (n=98) was shown to belong to the AVEN^{high} group and 38% (n=60) was shown to belong to the AVEN^{low} group. Specifically, only 24% (n=32) of AVEN^{high} patients were predicted as responders based on the TIDE tool (see FIG. 5A). In order to further demonstrate that AVEN is a potential genetic marker for predicting an immunotherapy response, additional data were retrieved from GSE207422 and GSE135222 along with immunotherapy treatment. In the GSE207422 cohort, major pathologic response (MPR), which is defined that less than 10% viable tumor cells remain by H&E staining, was defined as an immunotherapy responder, and NMPR was defined as a non-responder (NMPR). Among the 15 non-responders (NMPR), 10 patients were shown to have high levels of AVEN expression, and among the 9 total responders (MPR), 7 patients were shown to have low levels of AVEN expression. Additionally, AVEN^{high} patients showed a tendency of low progression-free survival after immunotherapy treatment in another cohort (GSE135222) (see FIGS. 5B and 5C).

### 5. Development of AVEN-Derived Genomic Model for Prognosis of Lung Adenocarcinoma

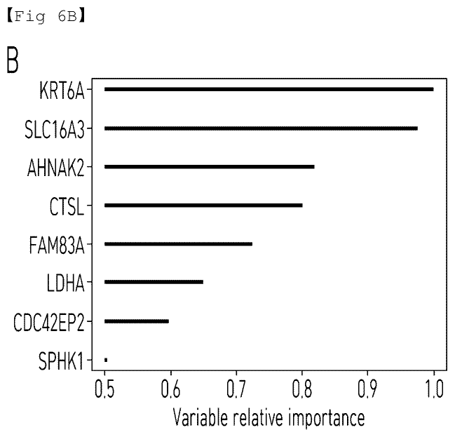

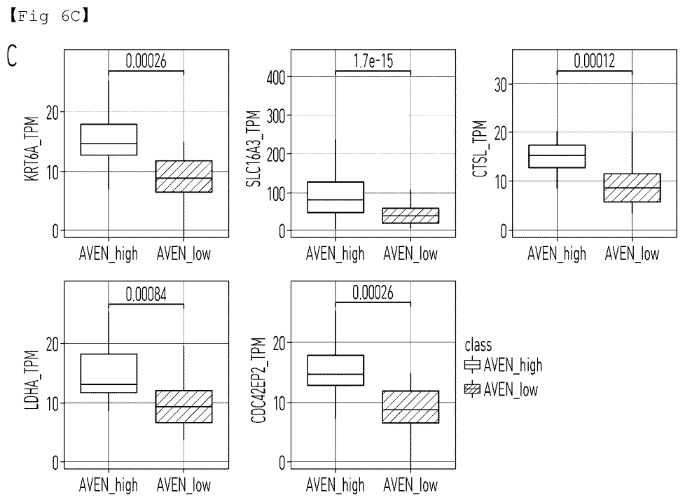

As a result of the examination of the effect of AVEN expression on various datasets using PrognoScan, while significant correlation with lung adenocarcinoma survival was observed in some cohorts, the significant prognostic effect of AVEN was shown to be limited in certain cohorts. Since AVEN-induced DEGs were shown to have a distinct effect on important oncogenic pathways, in the present invention, the prognostic effects of 838 DEGs were examined in detail using the Univariate Cox regression analysis. 305 Survival-related genes with p-value of <0.01 were determined to contribute to the survival of lung adenocarcinoma patients and are prognostic factors, and these genes were used as input data for the Random Survival Forest analysis (FIG. 6A).

As a result of calculating the relative importance of the above-mentioned prognostic genes and ranking these genes using the Random Survival Forest analysis, 8 genes with variable relative importance of 0.5 or greater were selected: KRT6A, SLC16A3, AHNAK2, CTSL, FAM83A, LDHA, CDC42EP2, and SPHK1 (FIG. 6B). Additionally, a Multivariate Cox regression model was developed using these genes, and in order to optimize the survival prediction model, Stepwise Forward analysis of the Multivariate-Cox regression was used to more specifically screen valuable prognostic genes, including KRT6A, SLC16A3, CTSL, LDHA, and CDC42EP2.

According to the model above, the risk score for each patient was identified as follows:

$$Risk score = h 0 ( t ) \times exp ( KRT 6 A \times 0.0002919 + SLC 16 A 3 \times 0.0045 + CTSL \times 0.0008009 + LDHA \times 0.00794 + CDC 42 EP 2 \times 0.0147 )$$

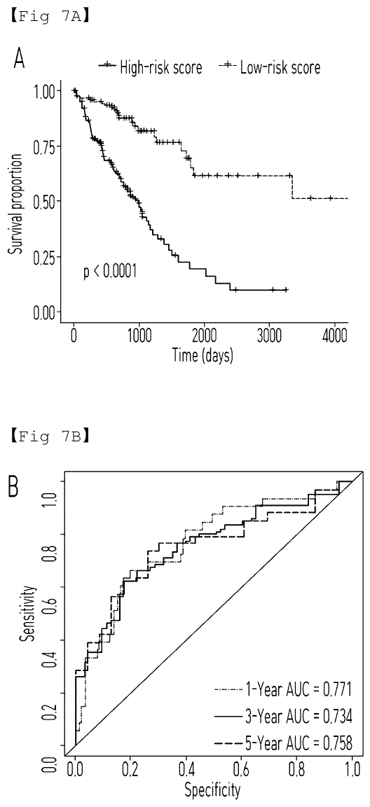

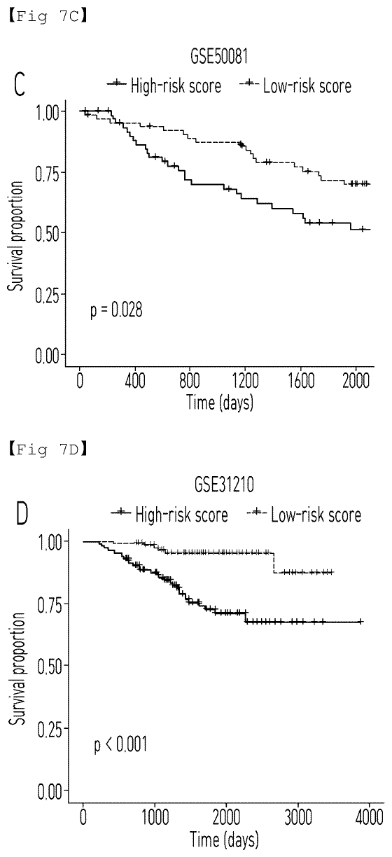

Based on the average risk score, lung adenocarcinoma patients were classified into high and low risk scores, Kaplan-Meier survival analysis showed that lung adenocarcinoma patients with a low risk score had a significant survival advantage over the population with a high risk score, thereby confirming that the AVEN-derived prognostic model is effective in predicting patient prognosis by performing time-dependent RPC curves (FIGS. 7A and 7B). The prognostic modes derived from AVEN were additionally evaluated using two independent external datasets, GSE50081 and GSE31210. As shown in FIGS. 7C and 7D, LUAD patients with lower risk scores showed higher survival rates, which means that the AVEN-derived prognostic model is effective and sufficient to predict LUAD survival.

In the above, the present invention has been described with a focus on preferred embodiments. Those having ordinary skill in the art to which the present invention belongs will be able to understand that the present invention can be implemented in a modified form without departing from the essential features of the present invention. Therefore, the disclosed embodiments should be considered from an illustrative aspect rather than a restrictive aspect. The scope of the present invention is indicated in the patent claims, not in the foregoing description, and all differences within the same scope should be construed as being included in the present invention.

What is claimed is:

1. A biomarker composition for lung adenocarcinoma diagnosis or prognosis prediction, comprising a substance that specifically binds to apoptosis, caspase activation inhibitor (AVEN) mRNA or its protein.

2. The composition of claim 1, wherein the substance is at least one selected from the group consisting of antibodies, aptamers, DNA, RNA, proteins, and polypeptides.

3. A kit for the diagnosis or prognosis prediction of lung adenocarcinoma comprising the composition of claim 1.

4. A method for providing information for the diagnosis of lung adenocarcinoma, comprising measuring the expression level of AVEN mRNA or its protein in a sample isolated from a subject for diagnosis.

5. The method of claim 4, further comprising determining that the subject for diagnosis has a higher probability of developing lung adenocarcinoma compared to the control group when the expression level is higher than that of the control group.

6. The method of claim 4, wherein the sample is selected from the group consisting of tissue, cells, blood, serum, plasma, saliva, and urine.

7. A method for prognosis prediction of lung adenocarcinoma, comprising:
a) classifying the gene expression data of patients with lung adenocarcinoma into upper and lower groups of AVEN gene according to the expression level of AVEN;
b) performing an analysis of differentially expressed genes (DEG) for each of a plurality of gene expression data in the two groups;
c) analyzing the correlation between the expression levels of the differentially expressed genes and survival of lung adenocarcinoma patients;
d) selecting KRT6A, SLC16A3, CTSL, LDHA, and CDC42EP2 genes as AVEN-associated genes; and
e) calculating a prognostic risk score for lung adenocarcinoma based on the expression levels of AVEN gene and the AVEN-associated genes.

8. The method of claim 7, wherein in step a) above, the upper group of AVEN gene shows the upper 25% expression level, and the lower group of AVEN gene shows the lower 25% expression level.

9. The method of claim 7, wherein step b) above is to select genes by examining molecular and cell signaling pathways associated with the AVEN gene through Spearman's Rank Correlation Test.

10. The method of claim 7, wherein step c) above is to select genes with a p value<0.01 as prognostic marker genes for lung adenocarcinoma associated with the survival of lung adenocarcinoma patients through Cox regression analysis.

11. The method of claim 10, further comprising calculating the relative importance of the prognostic marker genes for lung adenocarcinoma through random survival forest analysis, and selecting genes with an importance value of 0.5 or higher.

12. The method of claim 11, wherein the selected genes are selected from the group consisting of KRT6A, SLC16A3, AHNAK2, CTSL, FAM83A, LDHA, CDC42EP2, and SPHK1.

13. The method of claim 7, wherein the risk score of the step e) above is calculated by the following equation:
risk score=h0(t)×exp(KRT6A×0.0002919+SLC16A3×0.0045+CTSL×0.0008009+LDHA×0.007940+CDC42EP2×0.0147).
